(19) United States
(12) Patent Application Publication
(10) Pub. No.: US 2025/0177338 A1
(43) Pub. Date: Jun. 5, 2025

(54) COMPOSITION COMPRISING N-ACETYLCYSTEINE, VITAMIN D AND PREFERABLY GLUTATHIONE, USES THEREOF, AND PHARMACEUTICAL AND NUTRACEUTICAL COMPOSITIONS THEREOF

(71) Applicant: URIACH ITALY S.R.L., Assago (MI) (IT)

(72) Inventors: Lorenzo SECONDINI, Assago (MI) (IT); Mietta CATERA, Assago (MI) (IT); STEFANO LELLO, Assago (MI) (IT)

(21) Appl. No.: 18/854,918

(22) PCT Filed: Apr. 12, 2023
(86) PCT No.: PCT/IB2023/053745

§ 371 Date: Oct. 7, 2024

(30) Foreign Application Priority Data
Apr. 12, 2022 (IT) 102022000007229
Apr. 12, 2022 (IT) 102022000007238

(51) Int. Cl. A61K 31/198; A61K 31/592; A61K 31/593; A61K 38/06

(52) U.S. Cl. CPC A61K 31/198; A61K 31/592; A61K 31/593; A61K 38/063

(57) ABSTRACT

It is an object of the present invention a composition comprising N-acetylcysteine, or a pharmaceutically or food grade acceptable salt thereof or a derivative thereof, and a vitamin D, preferably vitamin D3 and/or vitamin D2, and preferably also glutathione or a derivative thereof. It is further an object of the present invention said composition for use in a therapeutic and/or non-therapeutic method of treatment of diseases, symptoms and/or disorders, for example, associated with brain aging, neuropathies, particularly peripheral neuropathies, and neurodegenerative diseases.

The present invention relates to a composition comprising N-acetylcysteine (NAC), vitamin D and, optionally, glutathione, and therapeutic and nontherapeutic uses thereof. The invention also relates to pharmaceutical and nutraceutical compositions thereof.

In more detail, it is an object of the present invention a composition comprising N-acetylcysteine, or a pharmaceutically or food acceptable salt thereof or a derivative thereof, a vitamin D, preferably vitamin D3 and/or vitamin D2, and, optionally, glutathione or a derivative thereof. It is further an object of the present invention said composition for use in a method of treatment, therapeutic and/or non-therapeutic, of diseases, symptoms and/or disorders, for example, associated with cerebral aging, particularly for use in a method of treatment of peripheral neuropathy.

According to a second aspect, the present invention relates to said composition for use in a method of treatment of neurodegenerative diseases.

Aging is an extremely complex multifactorial process characterized by a gradual continuous loss of physiological functions, particularly marked in the brain, where, however, related pathologies are not necessarily related to chronological aging.

Cognitive frailty is emerging as one of the greatest health threats of the 21st century. Life expectancy continues to rise but at the same time the prevalence of cognitive decline is increasing. This is becoming a serious social problem causing distress and increased costs for individuals, families, and health care systems.

A common hallmark of aging and aging-related diseases, particularly neurodegenerative diseases, is increased oxidative stress and concomitant failure of antioxidant defense systems. Formulations containing antioxidants have been known to be used in combination with symptomatic drugs to reduce oxidative stress and improve cognitive function in aging and aging-related diseases. In particular, the brain is highly susceptible to oxidative damage due to high concentrations of polyunsaturated fatty acids and transition metals that are involved in hydroxyl radical generation. In an adult brain, astrocytes are responsible in maintaining neuronal and synaptic function. Oxidative stress plays a key role in astrocyte loss, mainly due to highly active mitochondria metabolism. According to the literature, the brain has low catalytic activity and has low levels of protective antioxidant enzymes.

Patent application US 2007/116779 A1 describes a composition suitable for counteracting key attenuating factors specific to the degenerative processes occurring in Parkinson's disease. The composition comprises at least one substance that prevents 1-methyl-4-phenylpyridinium-induced toxicity through the promotion of ATP produced by anaerobic glycolysis ANAEROBIC (+), wherein said ANAEROBIC (+) further comprises one or more substances selected from the group consisting of pyruvic acid, succinic acid and/or oxaloacetate alone or in combination with vitamin B3, vitamin B3 derivatives, magnesium, acetyl-L-carnitine, alpha-ketoglutarate, phospho(enol)pyruvate, fructose, and fructose 1,6 bisphosphate.

U.S. Pat. No. 10,105,419 B2 describes a formulation for administration to type II diabetic individuals and healthy individuals to prevent the development of type II diabetes. The formulation comprises three components: at least one component consisting essentially of one or more proteins; at least one component comprising dandelion extract, milk thistle extract, phospholipids, ginger, taurine, curcuma extract, vitamin C, methionine, or combinations thereof; at least one component comprising magnesium, trimethylglycine, glutathione, N-acetyl-cysteine, Cordyceps sinensis extract, glutamine, alpha-lipoic acid, glycine, or combinations thereof; and an excipient.

LEMON J A et al: “A dietary supplement abolishe age-related cognitive decline in transgenic mice expressing elevated free radicalprocesses”; Experimental Biology and Medicine, Sage Publications Ltd., GB, Vol. 228, 1 Jan. 2003, describes a dietary supplement for the treatment of age-related decline in cognitive faculties.

The supplement contains several vitamins, such as vitamins C, B1, B3, B6, B12, D, E; acetyl-L-carnitine, alpha-lipoic acid, bioflavonoids, and co-enzyme Q10.

WO2022/043407 A1 describes a composition for the treatment of neurological diseases, specifically, brain stroke, multiple sclerosis, Parkinson's disease, Alzheimer's disease, and amyotropic lateral sclerosis as well as neurological conditions related to infection caused by SARS-Cov-2. The composition comprises either a pure active ingredient or a salt thereof. The active ingredient is selected from terpene (such as diterpene, triterpene, and sesquiterpene), quinone, an organic sulfide compound, and a vitamin (such as vitamin A, vitamin B, vitamins B1-B12, vitamin C, vitamin D, vitamin D3, Vitamin E; advantageously a combination of vitamins A, D3, and E).

A nutrient composition is described in international application WO2005/067972 A1 to enhance the immune system or physiological detoxification. The nutrient composition contains at least one antioxidant vitamin (such as vitamin C, vitamin B6, vitamin E, beta-carotene, and bioflavonoid complex), at least one antioxidant mineral (such as zinc and selenium), and at least three antioxidants with high antioxidant potency (such as alpha-lipoic acid, acetyl-L-carnitine, N-acetyl-cysteine, co-enzyme Q10, or glutathione).

WO 2010/082837 A1 describes the use of vitamin D3 to produce a drug for the transdermal treatment of neurogenic inflammation and neuropathic pain, including peripheral neuropathic pain.

WO 03/077900A1 refers to a method to prevent cancer and neurodegenerative diseases by administering an effective amount of N-acetyl-cysteine or a pharmacologically acceptable salt thereof.

Neurodegenerative diseases are a heterogeneous group of disorders characterized by progressive and selective neuronal death leading to degeneration of specific brain regions caused mainly by the natural aging process. The most common neurodegenerative diseases are Alzheimer's disease (AD) characterized by progressive loss of cognitive function and Parkinson's disease (PD) characterized by motor symptoms related to dopaminergic neuronal loss in the substantia nigra. Astrocyte functions are altered in the aging brain, and this contributes to reduced neuronal and synaptic function.

In any case, the body possesses defense mechanisms based on antioxidant actions; a group of enzymatic (e.g., superoxide dismutase, catalase, and glutathione reductase) or nonenzymatic (e.g., glutathione, melatonin, vitamins A, C, and E, and flavonoids) molecules that play an important role in maintaining cellular homeostasis and vitality. In some cases, the endogenous antioxidant system is not strong enough to counteract oxidative damage. For this reason, numerous studies have been conducted to investigate the effects of antioxidant dietary supplementation in aging or neurodegenerative diseases.

Finally, a typical mechanism of aging in a healthy subject is selective accumulation of iron that occurs in different brain regions and cell types. However, iron accumulation in specific brain regions, greater than that reported in healthy aging, occurs in many neurodegenerative diseases and is often associated with oxidative stress and cellular damage. Lipoic acid is known to be a potent chelator of bivalent metal ions, and vitamin D, particularly vitamin D3 and/or vitamin D2, is known to prevent iron accumulation-induced damage. However, the processes involved in aging-related iron accumulation and iron-induced inflammation in specific brain regions and cells are poorly understood to date.

Pharmaceutical compositions (drugs) or dietary supplements or nutraceuticals currently available on the market for the therapeutic and nontherapeutic treatment of diseases, symptoms, and/or disorders, for example, associated with brain aging, and in particular for the treatment of neurodegenerative diseases, such as Alzheimer's (AD) and/or Parkinson's (PD) disease, comprise lipoic acid and in some cases other compounds with co-acting function. For example, the Applicant has developed a composition comprising lipoic acid and vitamin D, specifically vitamin D3, described and claimed in WO2020/053754.

Lipoic acid has been shown to be very effective in counteracting diseases, symptoms, and/or disorders associated with cerebral aging, for the treatment of peripheral neuropathy and, in a second aspect of the present invention, neurodegenerative diseases. However, recent data seem to indicate that the use of lipoic acid in known dosages (generally greater than or equal to 800 mg/day) may cause side effects. According to prior art, lower dosages of lipoic acid do not appear to have the same therapeutic effect.

The technical problem that the present invention addresses and solves is to provide compositions that provide effective treatment, both therapeutic and non-therapeutic, of diseases, symptoms and/or disorders of various kinds, for example associated with cerebral aging, and/or for the treatment of neurodegenerative diseases, such as and example Alzheimer's (AD) and/or Parkinson's (PD), and/or for the treatment of peripheral neuropathy, in the absence or with reduced side effects, particularly in the absence of side effects associated with lipoic acid. In addition, the technical problem that the present invention addresses and solves is to provide a viable solution for the nontherapeutic treatment of subjects undergoing, or potentially undergoing, cerebral aging that slows down this process effectively and in the absence of side effects, particularly in the absence of side effects that can be associated with lipoic acid.

To overcome the said technical problems, the present invention provides a composition (pharmaceutical composition, nutraceutical composition, dietary supplement or medical device composition) comprising, as active ingredients, N-acetylcysteine or a pharmaceutically or food grade acceptable salt thereof or a derivative thereof, a vitamin of the D group, preferably vitamin D3 (cholecalciferol) and/or vitamin D2, and, preferably, also glutathione or a derivative thereof. Said composition is capable of effectively and rapidly treating numerous diseases, symptoms or disorders, for example, diseases, symptoms or disorders caused by or resulting from neuropathies, preferably peripheral neuropathies and, in a second aspect of the present invention, of neurodegenerative diseases, both in pathological and in healthy subjects (not yet defined as pathological).

In addition, the present invention provides a composition free of the side effects found in treatments of the prior art that is easy to prepare and cost-effective.

These purposes and others, which will be clear from the detailed description that follows, are achieved by the compositions and mixtures of the present invention through the technical features claimed in the united claims.

The Applicant, after intensive research and development activity, has found that administration of the composition according to the present invention allows maintaining or even improving the efficacy of known compositions, without the use of lipoic acid. And in fact, the composition of the invention is capable of effectively and rapidly treating numerous diseases, symptoms and/or disorders, for example caused by or resulting from cerebral (biological) aging, particularly neurodegenerative diseases and/or neuropathies, preferably peripheral neuropathies, and generally slowing down cerebral aging. Other examples of diseases, symptoms, and/or disorders effectively treatable by the composition of the invention include tumor, insulin resistance, metabolic disorders, obesity, thrombotic/thromboembolic diseases, ischemic episodes from surgery, transplantation bypass, endometriosis, infertility, polycystic ovary syndrome (PCOS), polyabortion (i.e., the occurrence of three or more consecutive miscarriages before the 20th week of pregnancy, each with a fetus weighing less than 500 grams) and inflammation. Moreover, the composition of the invention is effective in cases of comorbidity, that is, in the case of the simultaneous presence in the same subject of two or more diseases and/or disorders. In the context of the present invention, repeated abortion is defined as when, in a woman's obstetrical history, two consecutive episodes of abortion occur within the 20th week of pregnancy. Recurrent abortion is, on the other hand, defined as the presence of three or more consecutive episodes of miscarriage. Currently, it is generically referred to as polyabortion, and screening for this is implemented from the second consecutive episode of abortion.

The composition of the invention is, in addition, effective in cases of comorbidity, that is, in the case of the simultaneous presence in the same subject of two or more diseases and/or disorders. Advantageously, the composition of the invention exhibits marked antioxidant activity. Therefore, the composition of the invention can be used for the treatment of diseases, symptoms and/or disorders associated with oxidative stress and inflammation. The surprising pharmacological activity is due to the particular combination of the active ingredients in the composition, such as N-acetylcysteine, vitamin of the D group, preferably vitamin D3 and/or vitamin D2, and, preferably, also glutathione. In fact, these active ingredients act synergistically so that the effect of N-acetylcysteine is amplified allowing for equal or better results than known compositions containing lipoic acid, but without incurring the possible side effects due to the presence of this acid (and its dosage), as detailed in the experimental part.

### DESCRIPTION OF DRAWINGS

FIG. 1(a), FIG. 1(b), FIG. 1(c), FIG. 1(d): Cell viability measured with predigestion in the intestinal compartment (* p<0.05 vs control).

FIG. 2(a), FIG. 2(b), FIG. 2(c), FIG. 2(d): Assessment of absorption through intestinal epithelium (intra/extra ratio; * p<0.05 vs control).

FIG. 3: Assessment of hepatic uptake (* p<0.05 vs control).

FIG. 4: Assessment of biological activities at liver level: analysis of ERK activity (* p<0.05 vs control).

FIG. 5(a) and FIG. 5(b): assessment of biological activities at the liver level: analysis of CYP activity, especially CYP1A2 (FIG. 5(a)) and CYP3A4 (FIG. 5(b)) (* p<0.05 vs control).

FIG. 6: Evaluation at the Central Nervous System level: assessment of blood-brain barrier viability (* p<0.05 vs control).

FIG. 7: Evaluation at the Central Nervous System level: assessment of uptake at the blood-brain barrier.

FIG. 8: Central Nervous System evaluation: assessment of anti-oxidant properties (anti-oxidative stress) (* p<0.05 vs control). The test provides insight into whether or not there are pro-inflammatory effects and, consequently, whether there is any use in counteracting the effects of oxidative stress.

FIG. 9: Evaluation at the Central Nervous System level: assessment of anti-inflammatory activity (* p<0.05 vs control). The evaluation is performed on a marker related to one of the main mechanisms involved in the inflammatory process, namely TNFα.

FIG. 10(a) and FIG. 10(b): Evaluation at the Central Nervous System level: assessment of intracellular markers TAU (FIG. 10(a)) and APP (FIG. 10(b)) (* p<0.05 vs control).

FIG. 11: Evaluation at the Peripheral Nervous System level: assessment of mitochondrial metabolism (* p<0.05 vs control; ** p<0.05 vs NAC 12 mM+VitD) FIG. 12: Evaluation at the Peripheral Nervous System level: assessment of antioxidant properties (* p<0.05 vs control).

FIG. 13(a) and FIG. 13(b): Evaluation at the Peripheral Nervous System level: assessment of anti-inflammatory activity (* p<0.05 vs. control). The evaluation is performed on markers related to the two main mechanisms involved in the inflammatory response, namely TNFα (FIG. 13(a)) and IL1β (FIG. 13(b)).

FIG. 14(a) and FIG. 14(b): Evaluation at the Peripheral Nervous System level: intracellular markers p75NTR (FIG. 14(a)) and ERB3 (FIG. 14(b)) analysis (* p<0.05 vs control).

FIG. 15(a) and FIG. 15(b): Evaluation at the Peripheral Nervous System level: intracellular markers NRG1 (FIG. 15(a)) and MPZ (FIG. 15(b)) analysis (* p<0.05 vs control).

A composition (in short, composition of the invention) comprising:
(i) a mixture (in short mixture of the invention) comprising or alternatively consisting of:
(I) N-acetylcysteine or a pharmaceutically or food grade acceptable salt thereof or derivative thereof;
(II) a vitamin of the D group or a derivative thereof; and preferably
(III) glutathione or a derivative thereof;
said composition further comprising, when required:
(ii) at least one food or pharmaceutical grade additive and/or excipient.

N-acetylcysteine (in short NAC), also known by its IUPAC name 2R-acetamido-3-sulfanylpropanoic acid, is an N-acetylated derivative of the amino acid cysteine, and it is known for its antioxidant and mucolytic properties. It is also known to be used as an antidote in cases of excessive paracetamol intake.

In the context of the present invention, the term “N-acetylcysteine” refers interchangeably to N-acetylcysteine and/or N-acetylcysteinic acid.

“Derivative” of N-acetylcysteine, in the context of the present invention, is understood to mean a derivative of N-acetylcysteine known to the person skilled in the art to have antioxidant properties similar to N-acetylcysteine itself. Known N-acetylcysteine derivatives are, for example, N-acetylcysteinamide, described in K. Sunitha et al. N-Acetylcysteine amide: a derivative to 6ulfil the promises of N-Acetylcysteine; Free Radic Res. 2013 May; 47(5):357-67 (DOI: 10.3109/10715762.2013.781595) and the derivatives described in Shourung Liu et al. Identification of novel N-acetylcysteine derivatives for the treatment of hepatocellular injury; Medchemcomm. 2017 Dec. 1; 8(12): 2238-2247, doi: 10.1039/c7md00409; in particular compounds 6a [R-2-Acetamido-N-cyclohexyl-3-(methylthio)propanamide], 6b [R—N-Cyclohexyl-3-(methylthio)-2-propionamidopropanamide] and 7a [R-2-Acetamido-3-(methylthio)-N-phenylpropanamide].

In the context of the present invention, “vitamin of the D group” means a compound belonging to a group of fat-soluble pro-hormones of natural origin comprising vitamin D1 (ergocalciferol and lumisterol 1:1), vitamin D2 (ergocalciferol), vitamin D3 (cholecalciferol); vitamin D4 (dihydroergocalciferol), vitamin D5 (sitocalciferol), and mixtures thereof.

In the context of the present invention, “a vitamin of the D group, or a derivative thereof,” means a complex of at least one of the compounds that are part of the vitamins of the D group, for example, a compound selected from the protected derivatives, such as compounds that are part of the vitamins of the D group whose functional groups are protected with appropriate protecting groups, or metabolic precursors, known to the person skilled in the art.

Glutathione, IUPAC name (2S)-2-amino-5-[[(2R)-1-(carboxymethylamino)-1-oxo-3-sulfanylpropan-2-yl]amino]-5-oxopentanoic acid, is a tripeptide consisting of cysteine and glycine and glutamate. Glutathione has strong antioxidant properties.

In the context of the present invention, “glutathione or a derivative thereof” means glutathione in oxidized form (GSSG) or reduced form (GSH) or a mixture thereof.

In a preferred embodiment, in composition according to the invention, said (II) vitamin of the D group is vitamin D3 or cholecalciferol (CAS No. 67-97-0) and/or vitamin D2 or a derivative thereof as defined above.

In a preferred embodiment, the (i) mixture of the present invention comprises (I) N-acetylcysteine or a pharmaceutically or food grade acceptable salt thereof or a derivative thereof, (II) vitamin D3 and/or vitamin D2 or a derivative thereof, and (III) glutathione or a derivative thereof.

In the composition of the invention, according to the above embodiments, each component (I), (II) and (III) may be present in the amounts defined below.

Preferably, said (I) N-acetylcysteine is present in said (I) mixture of the invention in an amount by weight ranging from 10% to 90%, with respect to the total weight of the mixture, preferably from 30% to 85%, more preferably from 50% to 80%, e.g. 70%, 75%.

In embodiments, N-acetylcysteine is present in the composition of the invention in an amount by weight in the range between 100 mg and 1,000 mg, preferably between 150 mg and 800 mg, more preferably between 200 mg and 600 mg, even more preferably 300 mg.

Preferably the mixture is present in the composition of the present invention in an amount by weight from 10% to 90%, preferably from 20% to 80%, even more preferably from 30% to 70%, e.g. 40%, 50%, 60%.

Preferably, said (II) vitamin of the D group, preferably vitamin D3 (or cholecalciferol) and/or vitamin D2 is present in said (i) mixture of the invention in an amount by weight from 0.0001% to 10%, with respect to the total weight of the mixture, preferably from 0.001% to 3%, preferably from 0.01% to 1%, even more preferably from 0.05% to 0.5%. In embodiments said (II) vitamin of the D group, preferably vitamin D3 (or cholecalciferol) and/or vitamin D2 is present in the composition of the invention in an amount by weight from 2 μg to 1000 μg, with respect to the total weight of the composition, preferably from 3 μg to 500 μg, more preferably from 5 μg to 100 μg, even more preferably from 5 μg to 50 μg.

Preferably, said (III) glutathione is present in the mixture (i) of the invention in an amount by weight, with respect to the total weight of the mixture, from 15% to 85%, with respect to the total weight of the mixture, preferably from 20% to 75%, more preferably from 25% to 60%, and is present in the composition of the invention in an amount by weight from 10 mg to 1200 mg, with respect to the total weight of the composition, preferably from 25 mg to 1000 mg, more preferably from 50 mg to 800 mg, even more preferably from 75 mg to 600 mg, e.g. 100 mg, 150 mg, 200 mg, 250 mg or 300 mg.

Preferably, in the mixture contained in the composition of the invention, components (I) and (III) are present in a weight ratio N-acetylcysteine:glutathione of 3:1, preferably 2:1, even more preferably 1:1. For example, 600 mg of N-acetylcysteine and 200 mg of glutathione, or 400 mg of N-acetylcysteine and 200 mg of glutathione, or 200 mg of N-acetylcysteine and 100 mg of glutathione, or 200 mg of N-acetylcysteine and 200 mg of glutathione, or 100 mg of N-acetylcysteine and 100 mg of glutathione.

In embodiments, the mixture (i) according to the present invention comprises said (I) N-acetylcysteine in an amount by weight from 10% to 90%, said (II) vitamin of the D group, preferably vitamin D3 (or cholecalciferol) and/or vitamin D2 in an amount by weight from 0.001% to 10%, and said (III) glutathione in an amount by weight from 30% to 80%.

Preferably, components (I) and (III) are present in a weight ratio N-acetylcysteine:glutathione of 3:1, preferably 2:1, even more preferably 1:1.

In mixtures (I), and in compositions containing said mixtures (I), which are an object of the present invention, the weight ratio between N-acetylcysteine, or a salt or derivative thereof, and glutathione, or a salt or derivative thereof, is from 1:6 to 6:1, preferably it is from 1:5 to 5:1, more preferably it is from 1:4 to 4:1, even more preferably it is from 1:3 to 3:1, e.g. from 1:2 to 2:1, or 1:1.

For example, in mixtures (I) and compositions containing said mixture (I), which are an object of the present invention, the weight ratio between N-acetylcysteine, or a salt or derivative thereof, and glutathione, or a salt or derivative thereof, may be:
5:1 weight ratio, such as 50 mg/10 mg, or 150 mg/30 mg; or
4:1 weight ratio, such as 20 mg/5 mg, or 40 mg/10 mg, or 80 mg/20 mg; or
3:1 weight ratio, such as 30 mg/10 mg, or 90 mg/30 mg, 150 mg/50 mg, or 300 mg/100 mg; or
2:1 weight ratio, such as 100 mg/50 mg, or 200 mg/100 mg; or
1:1 weight ratio, such as 50 mg/50 mg, or 100 mg/100 mg, or 150 mg 150 mg.

In embodiments, the mixture (i) according to the present invention comprises said (I) N-acetylcysteine, or a salt or derivative thereof, and said (III) glutathione, or a salt or derivative thereof, in a weight ratio from 1:6 to 6:1, preferably ranging from 1:5 to 5:1, more preferably ranging from 1:4 to 4:1, even more preferably ranging from 1:3 to 3:1, e.g. 1:2 to 2:1, or 1:1.

In the context of the present invention, vitamin D, present in mixtures and compositions containing said mixtures, which are an object of the present invention, means vitamin D2, or vitamin D3, or mixtures thereof Vitamin D2 and vitamin D3.

The composition according to the present invention, in addition to the (i) mixture of the invention and optionally (ii) additives and/or excipients, may also include other active ingredients such as, but not limited to, anti-inflammatory agents, antioxidants, probiotics, antacids, vitamins of a group other than group D (e.g., vitamins A, B, C, E, and K), homotaurine, L-acetyl carnitine, plant extracts (e.g., withamina somnifera and bacopa), nervonic acid, mineral salts (e.g., salts of Zn, Mg, and others), and mixtures thereof.

It is an object of the present invention the composition of the invention as described above, wherein said composition is for use in a method of treatment, preventive and/or curative, of diseases, symptoms and/or disorders in a subject in need thereof.

In a preferred embodiment, the composition of the invention is for use in a method of treatment, preventive and/or curative, of diseases, symptoms and/or disorders associated with (biological) brain aging in a subject in need thereof; and/or a method of treatment, preventive and/or curative, of neurodegenerative diseases and/or symptoms or disorders associated with said neurodegenerative diseases; and/or in a method of treatment, preventive and/or curative of neuropathies, preferably peripheral neuropathies.

Neurodegenerative diseases are a diverse set of diseases of the central nervous system, sharing a chronic and selective process of cell death of neurons. Depending on the type of disease, neuronal deterioration can result in cognitive deficits, dementia, motor impairment, behavioral and psychological disorders.

Among the most well-known neurodegenerative diseases are:

Alzheimer's disease (AD); Parkinson's disease (PD); Huntington's disease; amyotrophic lateral sclerosis (ALS); frontotemporal dementia (FTD or presenile dementia); progressive supranuclear palsy (PSP or Steele-Richardson-Olszewski syndrome); Lewy body dementia (DLB); Creutzfeldt-Jakob disease (CJD) and Gerstmann-Sträussler-Scheinker disease (GSS).

Alzheimer's-Perusini's disease, also called Alzheimer's disease, presenile dementia of the Alzheimer's type, primary degenerative dementia of the Alzheimer's type, or simply Alzheimer's, is the most common form of progressively disabling degenerative dementia with onset predominantly at presenile age (over 65 years, but can occur earlier). In DSM-5 it is named as a major or mild neurocognitive disorder due to Alzheimer's disease.

Parkinson's disease, often referred to as, Parkinson's, idiopathic parkinsonism, primary parkinsonism, hypokinetic rigid syndrome, or agitating paralysis, is a neurodegenerative disease. The typical motor symptoms of the condition are the result of the death of cells that synthesize and release dopamine. Such cells are found in the substantia nigra, a region of the midbrain.

Huntington's disease is a genetic neurodegenerative disorder that affects muscle coordination and leads to cognitive decline and psychiatric problems. It typically onset during middle age; it is the most common genetically caused disease in clinical neurological pictures with abnormal involuntary movements.

Amyotrophic lateral sclerosis, or ALS, also called Lou Gehrig's disease or Charcot's disease or motor neuron disease, is a progressive neurodegenerative motor neuron disease that selectively affects motor neurons. ALS is characterized by muscle stiffness, muscle twitching and gradual weakness due to decreased muscle size. This results in difficulty with speech, swallowing, and eventually breathing.

The locution frontotemporal dementia (FTD) identifies a heterogeneous group of non-Alzheimer neurodegenerative dementias that are characterized by the presence of alterations in a degenerative-atrophic sense of the frontal and temporal brain lobes. This is an umbrella term for several pathologies or alternatively a disease with several variants; it is also called presenile dementia (as opposed to the usually more senile dementias such as vascular dementia) because of the onset usually around 50-60 years of age, and can often be confused with the early form of Alzheimer's disease; in the latter, however, it is the memory that is primarily impaired, whereas FTD is signaled at onset by sudden personality changes and behavioral-motor oddities in the absence of pre-existing psychiatric or neurological pathology, and only later by partial memory loss, along with increasing cognitive and motor impairment. The average survival is about 7-8 years. In DSM-5 it is named as major or mild frontotemporal neurocognitive disorder.

Progressive supranuclear palsy (PSP) or Steele-Richardson-Olszewski syndrome is a neurodegenerative disease first described in 1964. The neurodegeneration involves atrophy in the midbrain and other brain structures including the subthalamic nucleus, globus pallidus, the pons nuclei, and the black matter. PSP falls into a group of neurological disorders classified as “Parkinson-Plus” or “atypical Parkinson's” with symptoms reminiscent of Parkinson's disease, while other features are quite different.

Lewy body dementia (or DLB) is a neurodegenerative disease, a form of dementia similar to Alzheimer's disease but with earlier onset and often related to Parkinson's disease and Parkinsonian syndromes.

Creutzfeldt-Jakob disease (CJD), originally described in the 1920s by Hans Gerhard Creutzfeldt and Alfons Maria Jakob, is a rare neurodegenerative disease that leads to a fatal form of progressive dementia. The clinical syndrome is characterized by predominantly cortical polysectorial deficits with memory loss, personality changes, hallucinations, dysarthria, myoclonus, postural rigidity, and seizures.

Gerstmann-Sträussler-Scheinker syndrome (GSS) is a very rare and familial neurodegenerative disease with autosomal dominant inheritance, classified as transmissible spongiform encephalopathy (TSE).

In embodiments, the composition of the invention is for use in a method of treatment of the following neuropathies: diabetic neuropathy, carpal-tarsal-ulnar—radial tunnel syndrome, cervicobrachialgia, herpes zoster, intervertebral disc herniation, sciatic neuralgia-lumbosciatica, Guillain-Barré syndrome, toxic and deficiency neuropathies (such as, drug neuropathy, alcoholic neuropathy), infectious neuropathies (such as, HIV infection neuropathies, cytomegalovirus neuropathies).

Diabetes is the most frequent cause of diabetic neuropathy, a peripheral neuropathy that expresses itself with different clinical variants: symmetric polyneuropathy, focal neuropathy, and mixed forms. The incidence of diabetes mellitus (DM) is calculated to be around 6% in the general population; the prevalence of neuropathy is 7% at the onset of DM to rise to 25-30% after 20 years in patients with diabetes II.

Carpal tunnel syndrome is the most common entrapment peripheral neuropathy. In most cases it is idiopathic. Carpal tunnel syndrome is manifested by the onset of pain and paresthesias, especially at night, in the first three fingers of the hand and the lateral half of the fourth finger, as well as the lateral half of the palm. Over time, the pains and paresthesias also radiate to the forearm and sometimes to the arm.

Radial tunnel syndrome is compression of the radial nerve in the proximal portion of the forearm. Symptoms include forearm and elbow pain.

Tarsal tunnel syndrome involves pain in the ankle, foot, and sometimes the toes caused by compression or injury of the posterior tibial nerve.

Ulnar tunnel (or cubital tunnel) syndrome is compression or traction of the ulnar nerve at the elbow and palm. Symptoms include elbow pain and paresthesias in the distribution area of the ulnar nerve.

Cervicobrachialgia is caused by compression of a cervical nerve; it is characterized by pain originating from the posterior cervical region conditioning a functional limitation of flexion-extension and rotation movements of the neck, radiating to one or both upper limbs.

Herpes zoster, commonly called shingles, is a viral disease of the skin and nerve endings caused by the varicella-zoster virus. It is a ganglioradicular syndrome. The clinical picture of herpes zoster is characterized by pain, cutaneous, neurological, and broader infectious manifestations.

Intervertebral disc herniation occurs in the cervical and lumbar regions: because of the stresses to which these parts of the spine are subjected, the intervertebral discs are more likely to undergo degenerative processes. Thus, fractures of the outer fibrous ring and subsequent herniation of the disc are possible, which leaks out of the intervertebral space, compressing the nerve structures.

Sciatic neuralgia, or sciatica, is characterized by injury to the sciatic nerve at the radicular or trocular level predominantly in the sensitive fibers. The symptomatology consists mainly of intense, sometimes excruciating pain in the distribution area of L5-S1 i.e., in the lower gluteal region, posterior side of the thigh and leg.

Guillain-Barré syndrome is an acute polyradiculonevritis. It is the most frequent and best known cause of ascending paralysis: the clinical presentation is that of muscle weakness with acute or subacute onset in the lower limbs and ascending evolution with reaching nadir within four weeks.

The most frequently observed toxic neuropathies are those caused by:
metal poisoning, such as lead, mercury, thallium and arsenic
by drugs, such as antiblastics, chemotherapeutics, antiarrhythmics, anti-rheumatics

Infectious neuropathies encompass numerous clinico-pathological entities, among which are immunodeficiency virus infection neuropathies and cytomegalovirus neuropathies.

In the context of the present invention, the term composition for use in a method of treatment of peripheral neuropathy resulting from kidney disease does not include the treatment of inflammation in a population with chronic kidney disease (in short CKD) at the end-stage (end-stage renal disease or stage-5, in short ESRD) on hemodialysis (in short HD).

As discussed above, the composition of the invention can effectively and rapidly treat numerous diseases, symptoms and/or disorders. In addition to neurodegenerative diseases, and peripheral neuropathies, the composition of the invention can be used in the treatment of one or more diseases, symptoms, and/or disorders selected from tumor, insulin resistance, metabolic disorders, obesity, thrombotic/thromboembolic diseases, ischemic episodes from surgery, transplantation, bypass, endometriosis, infertility, polycystic ovary syndrome (PCOS), polyabortion (i.e., the occurrence of three or more consecutive miscarriages before the 20th week of pregnancy, each with a fetus weighing less than 500 grams) and inflammation. Moreover, the composition of the invention is effective in cases of comorbidity, that is, in the case of the simultaneous presence in the same subject of two or more diseases and/or disorders. In the context of the present invention, repeated abortion is defined as when, in a woman's obstetrical history, two consecutive episodes of abortion occur within the 20th week of pregnancy. Recurrent abortion is, on the other hand, defined as the presence of three or more consecutive episodes of miscarriage. Currently, it is generically referred to as polyabortion, and screening for this is implemented from the second consecutive episode of abortion.

Furthermore, the composition of the invention can be used for the treatment of diseases, symptoms and/or disorders associated with oxidative stress and inflammation. In other words, the particular combination of the three active ingredients in the composition, such as N-acetylcysteine, vitamin of the D group, preferably vitamin D3 and/or vitamin D2, and glutathione allows for the effective treatment of a wide variety of diseases, symptoms and/or disorders.

Moreover, the composition of the invention is effective in cases of comorbidity, that is, in the case of the simultaneous presence in the same subject of two or more diseases and/or disorders According to a preferred embodiment of the invention, the composition of the present invention comprises such amounts of (I), (II) and (III) as to ensure the administration to individuals in need thereof of a daily dose comprising:
from 50 mg to 2,000 mg, preferably from 100 mg to 2,000 mg, even preferably from 200 mg to 1,500 mg, more preferably from 300 mg to 1,000 mg, e.g. 150 g, or 250 mg, or 400 mg, or 500 mg, or 600 mg, or 700 mg, or 800 mg, or 900 mg, of (I) N-acetylcysteine;
from 2 micrograms (mcg) to 4,000 mcg, preferably from 2 micrograms (mcg) to 2,500 mcg, even preferably from 2 micrograms (mcg) to 1.000 mcg, more preferably from 50 mcg to 800 mcg, even more preferably from 100 mcg to 600 mcg, e.g., 200 mcg, or 300 mcg, or 400 mcg, or 500 mcg, or 1,000 mcg of vitamin D, preferably vitamin D3 and/or vitamin D2;
from 10 mg to 2,000 mg, preferably from 50 mg to 1,500 mg, more preferably from 100 mg to 1,200 mg, e.g., 200 mg, or 300 mg, or 400 mg, or 500 mg, or 600 mg, or 700 mg, or 800 mg, or 900 mg, or 1,000 mg, of (III) glutathione.

The composition of the invention can be administered to subjects in need thereof once or more times a day depending on the state of the subject and the amounts of (I) and (II) (and optionally (III)) included in the composition. Advantageously, the surprising efficacy of the formulation according to the invention allows for the same therapeutic or nontherapeutic effects as known formulations containing lipoic acid, without the use of such acid.

Further, the present invention describes a method of treatment, preventive and/or curative, of diseases, symptoms and/or disorders, wherein said treatment comprises administering the composition of the invention as defined above to a subject in need thereof. In embodiments, the present invention describes a method of treatment, preventive and/or curative, of diseases, symptoms and/or disorders associated with (biological) aging. In embodiments, the present invention describes a method of treatment, preventative and/or curative, of neurodegenerative diseases as defined above and/or symptoms or disorders associated with said neurodegenerative diseases. In embodiments, the present invention describes a method of treatment, preventively and/or curatively, of neuropathies, particularly peripheral neuropathies as defined above and/or symptoms or disorders associated with said neuropathies.

In of embodiments, the present invention describes a method of treatment, preventive and/or curative, of a disease, symptom, and/or disorder selected from tumor, insulin resistance, metabolic disorders, obesity, thrombotic/thromboembolic diseases, ischemic episodes from surgery, transplantation, bypass, endometriosis, polycystic ovary syndrome (PCOS) infertility, polyabortion, and inflammation.

In embodiments, the present invention describes a method of treatment, preventive and/or curative, of a disease, symptom and/or disorder associated with oxidative stress and inflammation.

It is an object of the present invention the non-therapeutic use of the composition of the invention as defined above for the non-therapeutic treatment of symptoms or disorders. In preferred embodiments, the non-therapeutic use of the composition of the invention is directed to the non-therapeutic treatment of symptoms or disorders associated with the brain (biological) aging in a subject in need thereof.

It is an object of the present invention the non-therapeutic use of the composition of the invention as defined above for the non-therapeutic treatment of slowing down the brain (biological) aging in a subject in need thereof.

Finally, it is an object of the present invention, a pharmaceutical composition, nutraceutical composition, nutritional supplement product or food product or food for special medical purposes or a medical device composition comprising or, alternatively, consisting of the composition of the present invention.

The term “medical device” in the context of the present invention is used in the meaning according to Italian Legislative Decree Feb. 24, 1997, No. 46, i.e., it means a substance or other product, used alone or in combination, intended by the manufacturer to be used in humans for the purpose of diagnosis, prevention, control, therapy or alleviation of a disease, which product does not exert its principal action, in or on the human body, for which it is intended, by pharmacological or immunological means or by metabolic process but whose function may be assisted by such means.

The composition of the present invention may be, as a non-limiting example, in a liquid form, such as solution, biphasic liquid system, suspension or syrup, in a semisolid form, such as gel, cream or foam, or in a solid form, such as powder, granules, flakes, aggregates, capsules, pills, bars and equivalent forms.

Preferably, the composition of the invention is for oral use, preferably in solid or liquid form, more preferably in solid form.

For the sake of clarity, to achieve the purpose of the present invention, the active ingredients of the mixture of the present invention (I), (II) and (III) can also be administered separately (preferably in a time interval from 5 minutes to 60 minutes, and preferably from 10 minutes to 30 minutes, and in any order but, preferably, (I), (II) and (III) are administered to a subject at the same time, even more preferably in a single composition to achieve a faster and more synergistic effect, and for ease of administration. Preferably, when the active ingredients (I), (II) and (III) are administered in a single composition, said single composition corresponds to the composition of the present invention.

“Method of treatment” in the context of the present invention means an intervention, comprising the administration of a substance, or mixture of substances, or combination thereof, having as its purpose the elimination, reduction/diminution, or prevention of a disease or pathology and symptoms or disorders thereof.

Unless otherwise stated, a statement that a composition “comprises” one or more components or substances means that other components or substances may be present in addition to the one, or those, specifically stated.

The composition of the present invention is intended indifferently for human or veterinary use, that is, as a preparation to be applied to animals with the uses and methods known to the person skilled in the art.

As illustrated in the experimental part below, the compositions of the present invention are found to be suitable for the effective treatment, both therapeutic and non-therapeutic, of diseases, symptoms and/or disorders, particularly associated with brain (biological) aging, specifically for the treatment of neurodegenerative diseases as defined above and/or symptoms or disorders associated with said neurodegenerative diseases, particularly in the absence of relevant side effects. These results support the concept of synergistic effect of the individual components of the composition of the invention acting synergistically in the treatment of astrocytes under oxidative stress conditions and allows preventing oxidative damage in astrocytes dependent on Fe³⁺ accumulation, as detailed in the experimental part.

FRn preferred embodiments of the present invention are shown below.

FR1. A composition comprising:
(i) a mixture that comprises or alternatively consists of:
   (I) N-acetylcysteine or a pharmaceutically or food grade acceptable salt thereof or derivative thereof;
   (II) a vitamin of the D group or a derivative thereof; and, preferably,
   (III) glutathione or a derivative thereof; and optionally,
(ii) at least one food or pharmaceutical grade additive and/or excipient.

FR2. The composition according to FR1, wherein said (II) vitamin of the D group is a vitamin D3 and/or vitamin D2 or cholecalciferol or a derivative thereof.

FR3. The composition according to FR1 or FR2, wherein said mixture (i) comprises or alternatively consists of:
said (I) N-acetylcysteine in an amount by weight ranging from 10% to 90%, preferably from 30% to 70%, with respect to the total weight of the mixture;
said (II) vitamin of the D group in an amount by weight ranging from 0.0001% to 10%, preferably from 0.001% to 3%, with respect to the total weight of the mixture;
said (III) glutathione in an amount by weight ranging from 15% to 85%, preferably from 20% to 75%, with respect to the total weight of the mixture.

FR4. The composition according to any one of FR1-3, wherein said composition is for use in a method of treatment, preventive and/or curative, of a disease, symptom, and/or disorder associated with brain aging in a subject in need thereof.

FR5. The composition for use according to any one of FR1-3, wherein said composition is for use in a method of treatment, preventive and/or curative, of a neurodegenerative disease and/or symptoms or disorders associated with said neurodegenerative disease.

FR6. The composition for use according to any one of FR1-3, wherein said composition is for use in a method of treatment, preventive and/or curative, of peripheral neuropathy and/or symptoms or disorders associated with said peripheral neuropathy.

FR7. The composition for use according to FR5, wherein said neurodegenerative disease is selected from: Alzheimer's disease (AD), Parkinson's disease (PD), Huntington's disease, amyotrophic lateral sclerosis (ALS), frontotemporal dementia (FTD or presenile dementia), progressive supranuclear palsy (PSP or Steele-Richardson-Olszewski syndrome), Lewy body dementia (DLB), Creutzfeldt-Jakob disease (MCJ), and Gerstmann-Sträussler-Scheinker disease (GSS).

FR8. The composition for use according to FR6, wherein said peripheral neuropathy is selected from: diabetic neuropathy, carpal, tarsal, ulnar, radial tunnel syndrome, cervicobrachialgia, herpes zoster, herniated intervertebral disc, sciatic neuralgia or lumbosciatica, Guillain-Barré syndrome, toxic and/or deficiency neuropathies, preferably drug neuropathy and alcoholic neuropathy, and infectious neuropathies, preferably HIV infection neuropathy and cytomegalovirus neuropathy.

FR9. The composition according to any one of FR1-3, wherein said composition is for use in a method of treatment, preventive and/or curative, of an associated disease, symptom and/or disorder in a subject in need thereof, wherein said disease, symptom and/or disorder is selected from: tumor, insulin resistance, metabolic disorders, obesity, thrombotic/thromboembolic diseases, ischemic episodes from surgery, transplantation, bypass, endometriosis, infertility, polycystic ovary syndrome (PCOS), polyabortion, inflammation, and oxidative stress.

FR10. The composition for use according to any one of the FR1-9, wherein said composition comprising (I) and (II) and, preferably, (III) is administered in an effective amount, at least once or twice a day, so as to ensure the administration to subjects in need thereof of daily dosages in the ranges:
from 50 mg to 2,000 mg, preferably from 100 mg to 2,000 mg, even preferably from 200 mg to 1,500 mg, more preferably from 300 mg to 1,000 mg, e.g. 150 mg, or 250 mg, or 400 mg, or 500 mg, or 600 mg, or 700 mg, or 800 mg, or 900 mg, of (I) N-acetylcysteine;
from 2 micrograms (mcg) to 4,000 mcg, preferably from 2 micrograms (mcg) to 2,500 mcg, even more preferably from 2 micrograms (mcg) to 1.000 mcg, more preferably from 50 mcg to 800 mcg, even more preferably from 100 mcg to 600 mcg, e.g., 200 mcg, or 300 mcg, or 400 mcg, or 500 mcg, or 1,000 mcg of vitamin D, preferably vitamin D3 and/or vitamin D2;
from 10 mg to 2,000 mg, preferably from 50 mg to 1,500 mg, more preferably from 100 mg to 1,200 mg, e.g., 200 mg, or 300 mg, or 400 mg, or 500 mg, or 600 mg, or 700 mg, or 800 mg, or 900 mg, or 1,000 mg, of (III) glutathione.

FR11. The composition for use according to any one of FR1-11, wherein said composition is for oral use in said subject in need thereof.

FR12. Non-therapeutic use of the composition according to any one of FR1-3, wherein said composition is for use in a method of non-therapeutic treatment of a symptom or disorder, preferably associated with brain aging in a subject in need thereof.

FR13. Non-therapeutic use of the composition according to any one of FR1-3, wherein said composition is for use in a method of non-therapeutic treatment of slowing down brain aging in a subject in need thereof.

### Experimental Evidence Materials and Methods

### Cell Cultures

### Gastric Cell Line

The GTL-16 cell line, donated by the Laboratory of Histology, University of Eastern Piedmont (Italy), is a clonal line derived from a poorly differentiated gastric carcinoma cell line widely used as a model of gastric epithelial cells. Cells were cultured in Dulbecco's Modified Eagle medium (DMEM, Merck Life Science, Rome, Italy) supplemented with 10% fetal bovine serum (Fetal Bovine Serum, FBS), 1% penicillin-streptomycin, in an incubator at 37° C., 5% CO₂. 1×10⁴ cells were used to explore cell viability by MTT.

### Intestinal Cell Line

The Caco-2 cell line, provided by the American Type Culture Collection (ATCC), was cultured in Dulbecco's modified Eagle's/F-12 Ham Nutrient medium (DMEM-F12, Merck Life Science, Rome, Italy) containing 10% FBS, L-glutamine 2 mM and 1% penicillin-streptomycin, in an incubator at 37° C., 5% CO₂. This cell line is being used to develop a widely accepted (by EMA and FDA) experimental model to predict the absorption, metabolism, and bioavailability of drugs and xenobiotics after oral intake. Cells were used at passage numbers between 26 up to 32 (Cell cultures grow in plate, confluence refers to the space occupied by the cells. Passage, on the other hand, refers to the number of times cells were moved from one plate to a new plate.) To preserve the physiological balance between paracellular permeability and transport properties. This cell line was co-cultured with HT29-MTX calyciform cells, which exhibit intestinal barrier properties in terms of paracellular transport and relative efflux. This model makes it possible to in vitro recreate the intestinal epithelium in the most physiological way possible. The co-culture was seeded at a density of 7:3 (Caco-2:HT29-MTX) on 6.5-mm Transwell® with 0.4-μm pore size polycarbonate membrane inserts (Merck Life Science, Italy) in a 24-well plate to perform uptake studies.

### Hepatic Cell Line

Human epithelial cells (HepG2) were purchased from ATCC, and cultured in DMEM supplemented with 10% FBS, L-glutamine 2 mM and 1% penicillin-streptomycin, at 37° C., 5% CO2. The cells used in these experiments had a 90-95 passage range, and after reaching 80-90% confluence, 2×10⁵ cells/well were grown in 96-well plates for a cytotoxicity assay using a trypan blue assay; thereafter, 1×10⁴ cells were used for permeability detected by 0.04% fluorescein (Merck Life Science, Italy) by sinusoid transwell, and finally, 1×10⁶ on a 6-well plate to analyze activated intracellular pathways by Western blot analysis.

### Neuronal Cell Line and BBB (“Blood-Brain Barrier”)

Primary cultures of mouse astrocytes were prepared from both male and female C57BL/6 mouse pups, following a standard technique described elsewhere according to the National Guidelines for the Use and Care of Laboratory Animals. Briefly, within 24 h after birth, the pups were euthanized, and the cortices were mechanically dissected and digested. The cell suspension was centrifuged at 800 rpm for 5 min. Pelleted cells were resuspended in neuronal basal medium (Sigma-Aldrich, Milan, Italy) supplemented with 5% FBS, 1% penicillin/streptomycin and L-glutamine 2 mM, plated in multiple wells and maintained in culture for 6 days before treatment. Astrocytes should be separated from microglia and oligodendrocyte precursor cells by shaking, as reported in the literature. For experiments, 1×10⁴ cells were plated on a 96-well plate to study cell viability by MTT assay and ROS production by colorimetric assay; 1×10⁶ cells were plated on a 6-well plate to analyze TNFa, IL1b and molecular pathways by Western blot or ELISA analysis. Next, in order to recreate the Blood-Brain Barrier (BBB), astrocytes were co-cultured with human umbilical vein endothelial cells (HUVECs) according to methods reported in the literature. HUVECs were purchased from ATCC®. Cells were cultured in EGM media (Lonza, Basel, Switzerland) supplemented with 10% FBS, 1% penicillin/streptomycin and glutamine 2 mM at 37° C. in a humidified atmosphere with 95% air, 5% CO2. In summary, to create the BBB barrier, 4×10⁴ astrocytes/cm² were plated on the basolateral side of 6.5-mm inverted Transwells with a polyester membrane with a pore size of 0.4 μm and allowed to set for 4 h. The Transwells were then placed in the normal orientation and the cells allowed to grow for 48 h. After this time had elapsed, 1×10⁵ HUVEC cells/cm² were plated into the apical compartment. The inserts were then placed in a 24-well plate. After 7 days of culture, Transwells were processed, and permeability studies were performed.

### Co-Culture of the Peripheral Nervous System

In order to recreate a peripheral nervous system in vitro, a myelinating co-culture system was used with an NSC-34 motor neuron-like cell line and an IFRS1 adult rat Schwann cell line according to a protocol reported in the literature. IFRS1 cells were seeded on 100-mm plastic discs (Greiner Bio-One GmbH, Frickenhausen, Germany, 664-160-013) at an approximate density of 2×10⁴/cm² and maintained in DMEM containing 5% FBS, recombinant human eregulin-β (EMD Millipore, Billerica, MA, USA) 20 ng/mL, forskolin (Sigma, St. Louis, MO, USA) 5 μM and antifungal antibiotic solution (penicillin 100 U/mL, streptomycin 100 μg/mL and amphotericin 250 ng/mL; Sigma). NSC-34 cells were seeded on 100-mm plastic discs at an approximate density of 1×10⁴/cm² and maintained in DMEM containing antibiotic antifungal solution and 5% FBS. At semi-confluence, cells were detached from the discs using 0.05% trypsin/EDTA 0.53 mM solution, suspended in DMEM/F12 Ham containing 5% FBS in a 50 mL polypropylene conical tube, and reseeded on Aclar fluorocarbon coverslips coated with poly-L-lysine/laminine, 12-well culture plates coated with type I collagen, and 2-well glass chamber slides coated with type I collagen at an approximate density of 2×10³/cm². Cells were maintained in DMEM/F12 containing 1% FBS, 1% MEM nonessential amino acids and 10 ng/mL recombinant rat brain-derived neurotrophic factor (BDNF) for 5-7 days. When elongation of neurites from most NSC-34 cells was observed under a phase-contrast microscope, the cells were incubated for 12-16 h with DMEM/F12 containing 5% FBS and mitomycin C 1 μg/mL. NSC-34 cells were then rinsed twice with 5% DMEM/FBS, and co-cultured with IFRS1 cells, which had been detached from the discs using trypsin/EDTA and suspended in 5% DMEM/FBS at an approximate density of 1×10⁵/mL. The cell density ratio of NSC-34:IFRS1 was adjusted from 1:7 to 1:10. The co-cultured cells were then fed twice weekly in DMEM containing 5% FBS, ascorbic acid 50 μg/mL, BDNF 10 ng/mL and recombinant rat ciliary neurotrophic factor 10 ng/mL, and maintained for up to 28 days.

### Experimental Protocol

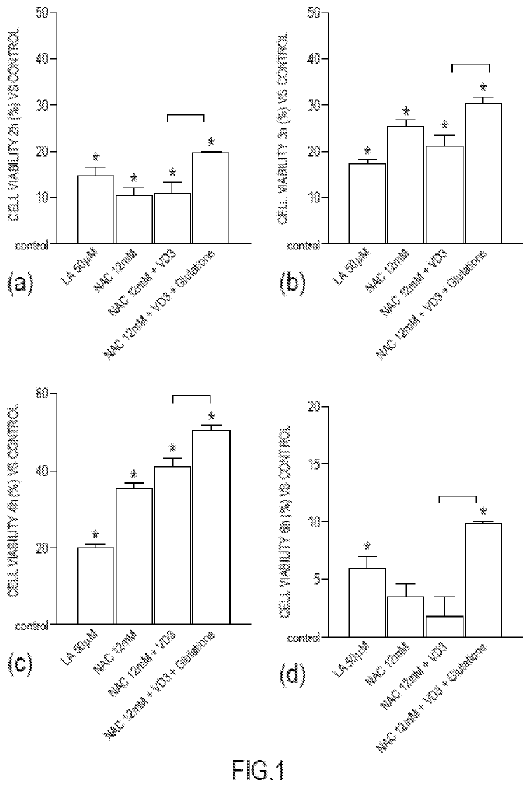

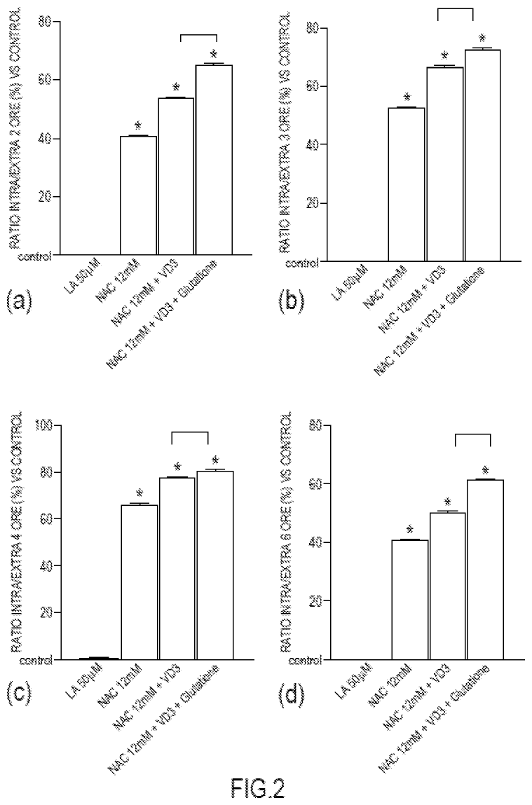

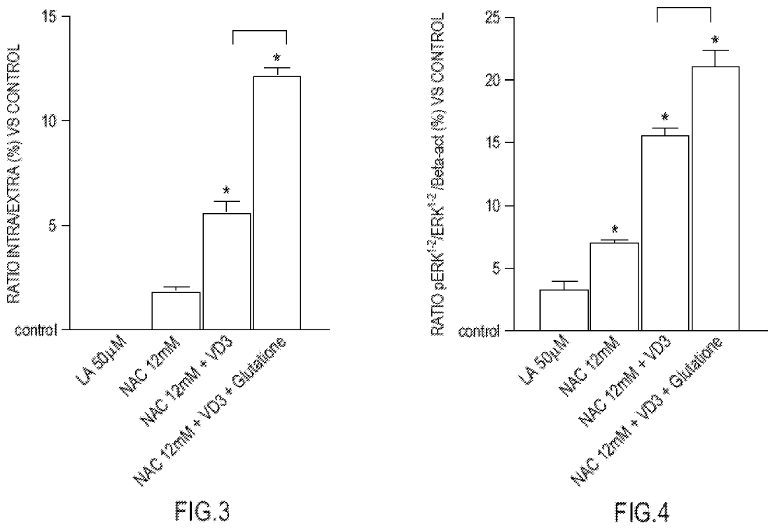

Cells were used to study different biological aspects of N-acetylcysteine, alone and combined with vitamin D3 and/or vitamin D2 and glutathione, and lipoic acid (50 μM), involved in many body districts. Firstly, the role of N-acetylcysteine (12 mM), alone and combined with vitamin D3 and/or vitamin D2 100 nM and glutathione 5 mM, on a gastric cell line was examined in a time course study to exclude any cytotoxic effect (from 2 hours to 6 hours—FIG. 1) in addition, permeability and absorption rate (from 2 h to 6 h—FIG. 2) were determined by evaluating the apparent permeability coefficient (Papp) and N-acetylcysteine concentration using an intestinal barrier model. Second, the hepatic metabolism of N-acetylcysteine (12 mM) alone and combined with vitamin D3 and/or vitamin D2 100 nM and glutathione 5 mM was examined (FIG. 3). In this context, a HepG2 cell line was used to analyze the major intracellular pathways involved in inflammation and N-acetylcysteine metabolism. Additional experiments were also performed on the BBB to evaluate the antioxidant and anti-inflammatory properties of N-Acetylcysteine (12 mM), alone and combined with vitamin D3 and/or vitamin D2 100 nM and glutathione 5 mM, by analyzing cell viability, ROS production, inflammatory marker and the main intracellular pathway involved in brain aging for 24 hours; in addition, N-Acetylcysteine dosage determination and permeability through the BBB were evaluated. Finally, the same experiment conducted on the BBB was also performed on a co-culture of NSC34/IRF1 in order to evaluate the biological effect of N-Acetylcysteine, alone and combined with vitamin D3 and/or vitamin D2 100 nM and glutathione 5 mM, on the peripheral nervous system.

The following experiments were conducted by treating cells with alpha-lipoic acid (50 μM).

### MTT

In this test, we can refer to all images where “cell viability” is reported as this test assesses cell viability.

A MTT-based in vitro toxicology assay kit (Sigma-Aldrich) was used on a 96-well plate to determine cell viability after each stimulation as previously described [Molinari C, Morsanuto V, Ghirlanda S, et al. Role of Combined Lipoic Acid and Vitamin D3 on Astrocytes as a Way to Prevent Brain Ageing by Induced Oxidative Stress and Iron Accumulation. Oxid Med Cell Longev. 2019; 2019:2843121. Published 2019 Feb. 28. doi:10.1155/2019/2843121]. At the end of stimulation, cells were incubated with 1% MTT dye for 2 h at 37° C. in an incubator, 5% CO₂ and 95% humidity, and then purple formazan crystals were dissolved in an equal volume of MTT solubilization solution. Cell viability was determined by measuring the absorbance at 570 nm with a correction at 690 nm, by a spectrometer (VICTOR X4, multiwell plate reader) and calculated by comparing the results with the control.

### ROS Production

The rate of superoxide anion release was used to examine the ROS produced by astrocytes after stimulations. After treatment, 100 μl of cytochrome C was added to all samples (treated or not), and in another sample, 100 μl of superoxide dismutase was also added for 30 min in an incubator (all substances were from Sigma-Aldrich). Absorbance was measured by a spectrometer (VICTOR X4, multiwell plate reader), at 550 nm, and O₂ was expressed as mean±SD of nanomoles per reduced cytochrome C per microgram of protein versus control on percent (%).

### In Vitro Permeability

It is well known that TEER is used to determine that Transwells or co-cultures reach a proper maturational state that allows experimental assays to begin, as well as to eventually verify their biological integrity. The data are usually used only in publications or if there is no possibility of performing specific assays. In this case we indicated it because it is part of experimental practice [Galla R, Grisenti P, Farghali M, Saccuman L, Ferraboschi P, Uberti F. Ovotransferrin Supplementation Improves the Iron Absorption: An In Vitro Gastro-Intestinal Model. Biomedicines. 2021; 9(11):1543; Srinivasan B, Kolli A R, Esch M B, Abaci H E, Shuler M L, Hickman J J. TEER measurement techniques for in vitro barrier model systems. J Lab Autom. 2015 April; 20(2):107-26. doi: 10.1177/2211068214561025].

In all analyzed compartments, transepithelial electrical resistance (TEER) was measured using an epithelial voltohmeter (EVOM3; WPI, Berlin, Germany). Measurements were performed at each culture medium replacement according to the manufacturer's instructions. In addition to TEER measurements, Fluorescein was added to the apical compartment of the Transwell system to determine the rate of cell layer uptake. This was performed as previously published [Molinari C, Morsanuto V, Ruga S, et al. The Role of BDNF on Aging-Modulation Markers. Brain Sci. 2020; 10(5):285. Published 2020 May 9. doi:10.3390/brainsci10050285; Hoffmann P, Burmester M, Langeheine M, Brehm R, Empl M T, Seeger B, et al. (2021) Caco-2/HT29-MTX co-cultured cells as a model for studying physiological properties and toxin-induced effects on intestinal cells. PLoS ONE 16(10): e0257824. https://doi.org/10.1371/journal. pone.0257824]. Cell-free negative controls were tested to exclude any influence.

### TNFα

TNFα concentration on basolateral environment was determined using the TNF-α ELISA kit (Sigma, Milan, Italy) according to the manufacturer's instructions. The absorbance of each well was measured after addition of stop solution at 450 nm using a plate reader (VICTOR X4, multiwell plate reader).

### IL1β

Basolateral co-culture medium was collected for IL-1β quantification with the ELISA kit for IL-1β (R&D systems, MN) according to the manufacturer's instructions. Chemiluminescent absorbance was determined using a microplate reader at 450 nm with correction at 570 nm. IL-1β was quantified by correlating sample readings to the generated standard curve.

### ERK Activation Assay

ERK/MAPK activity was measured by InstantOne™ ELISA (Thermo Fisher) on cell lysates following the manufacturer's instructions. The strips were measured by a spectrometer (VICTOR X4, multiwell plate reader) at 450 nm. Results were expressed as mean absorbance (%) compared with control.

### Quantification of Amyloid Precursor Protein (APP)

Quantification of amyloid precursor protein (APP) was measured by the Amyloid Beta A4 protein ELISA kit (Sigma-Aldrich) on cell supernatants following the manufacturer's instructions. APP concentration was determined by measuring absorbance by a spectrometer (VICTOR X4, multiwell plate reader) at 450 nm and calculated by comparing the results with the APP standard curve.

### Western Blot

Cells were washed and subsequently lysed in cold Complete Tablet buffer (Roche) supplemented with sodium orthovanadate 2 mM, phenylmethanesulfonyl fluoride 1 mM (PMSF; Sigma-Aldrich) and 1:100 Protease Inhibitor Cocktail (Sigma-Aldrich) mixture. From each lysate, 35 micrograms of protein were treated with SDS and loaded onto SDS-PAGE gels, and polyvinylidene difluoride (PVDF) membranes (GE Healthcare) were incubated overnight at 4° C. with a specific primary antibody: anti-CYP1A2 (1:250, Santa Cruz), anti-CYP3A4 (Thr198, 1:250, Santa Cruz), anti-TRKb (1:250, Santa Cruz), anti-pTau (1:250, Santa Cruz), anti-p75 (1:250, Santa Cruz), anti-ErbB3 (1:250, Santa Cruz), anti-NRG1 (1:250, Santa Cruz), anti-P0 (1:250, Santa Cruz), and anti-GAPDH (1:250, Santa Cruz). Protein expression was normalized and verified by detection of β-actin (1:5000; Sigma-Aldrich) and expressed as mean±SD (% vs. control).

### Statistical Analysis

The reported data were obtained from at least five independent experiments performed in triplicates for each experimental protocol and analyzed using Prism GraphPad statistical software. The reported results are expressed as means±SD using one-way ANOVA with subsequent Bonferroni post hoc test for statistical analysis. Values of p<0.05 were considered statistically significant.

The tests were carried out using:
lipoic acid 50 μM (referred to as “LA” in the figures);
N-acetylcysteine (or N-acetylcysteinic acid) 12 mM (referred to as “NAC” in the figures);
Vitamin D 100 nM (referred to as “VitD” or “VD3” in the figures);
glutathione 5 mM (referred to as “G” in the figures).

### Results

FIGS. 1(a)-(d), show the results of a cell viability assay measured in pre-digested model in the intestinal compartment at different times. Specifically, results observed after 2 hours (FIG. 1(a)), 3 hours (FIG. 1(b)), 4 hours (FIG. 1(c)) and 6 hours (FIG. 1(d)) are shown. As can be seen, the kinetics show a physiological effect that seems to peak around 4 hours. The presence of glutathione (G) contributes to increased cell viability.

FIGS. 2(a)-(d), show the results of an absorption test, conducted to assess the proportion of NAC absorbed through the intestinal epithelium (intra/extra ratio), at different times. Specifically, the results observed after 2 hours (FIG. 2(a)), 3 hours (FIG. 2(b)), 4 hours (FIG. 2(c)) and 6 hours (FIG. 2(d)) are shown. The test showed that the ratio of the extra to intra portion of N-acetylcysteine confirms what has been observed on kinetics and shows that combined with VitD and Glutathione at 3 hours increases absorption, reaching a maximum absorption at 4 hours.

FIG. 3 shows the results of a hepatic absorption test. The test showed that the combined compound (NAC+VitD) is better absorbed at hepatic level than NAC and lipoic acid alone. It was also observed that the presence of glutathione further increases hepatic absorption.

Hepatic uptake of both lipoic acid and NAC was analyzed using the same method for both and analyzing their respective wavelengths as reported in the literature [Molinari C, Morsanuto V, Ghirlanda S, Ruga S, Notte F, Gaetano L, Uberti F. Role of Combined Lipoic Acid and Vitamin D3 on Astrocytes as a Way to Prevent Brain Ageing by Induced Oxidative Stress and Iron Accumulation. Oxid Med Cell Longev. 2019 Feb. 28; 2019:2843121; Ruru Li, Xiaoyu Huang, Guolin Lu a and Chun Feng; A fluorescence and UV/vis absorption dual-signaling probe with aggregation-induced emission characteristics for specific detection of cysteine. RSC Adv., 2018, 8, 24346-24354. DOI: 10.1039/C8RA03756F]. The difference is probably due to the different hepatic metabolization of the compounds.

FIG. 4 shows an analysis of ERK activity, following different stimulations. The results obtained show that both major biological and antioxidant activities are conserved in the liver, with no toxic effect. It was also observed that the presence of glutathione further enhances the conservation of these biological activities.

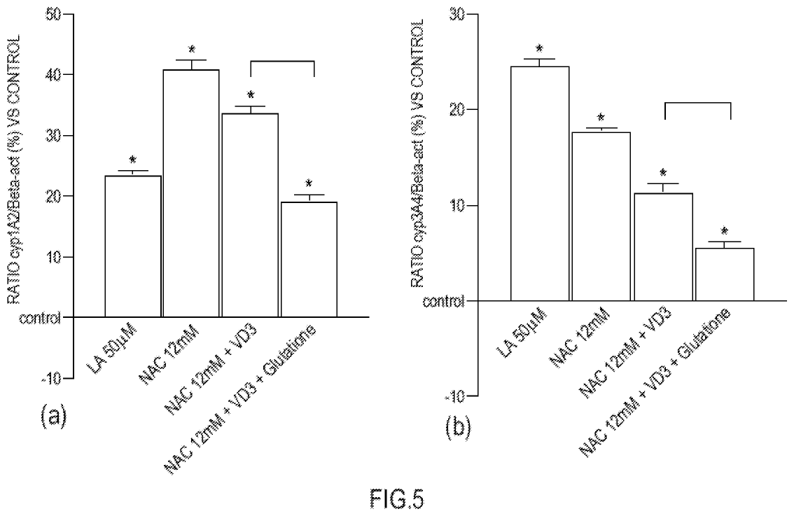

FIG. 5(a) and FIG. 5(b) show the results of a test conducted to evaluate the effect of different compounds and compositions on CYP1A2 (FIG. 5(a)) and CYP3A4 (FIG. 5(b)) cytochromes.

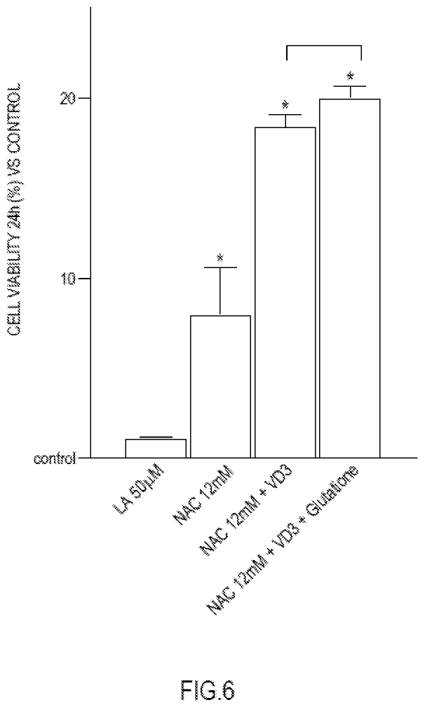

FIG. 6 shows the results of an experiment evaluating the viability of the blood-brain barrier following different stimulations for 24 hours. The test makes it possible to confirm that the product used reaches functional beyond the blood-brain barrier without side effects. The results obtained show that none of the combinations tested cause cytotoxic effects in the brain. The better efficacy of NAC+VitD compared with NAC alone and LA alone is confirmed. It was also observed that the presence of glutathione further enhances this effect.

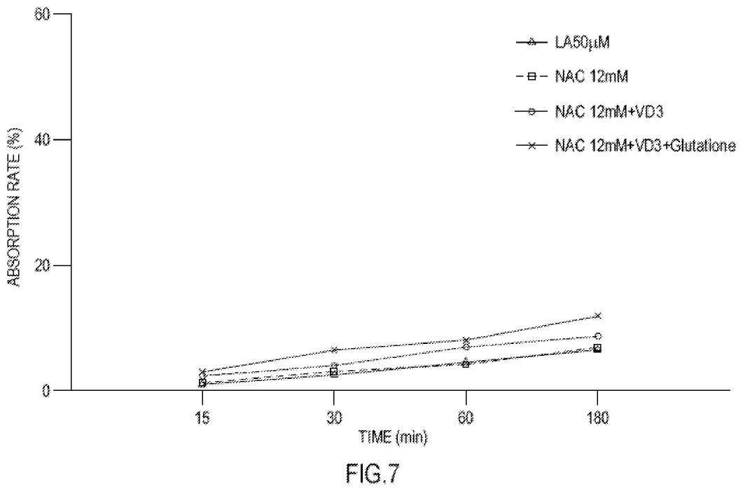

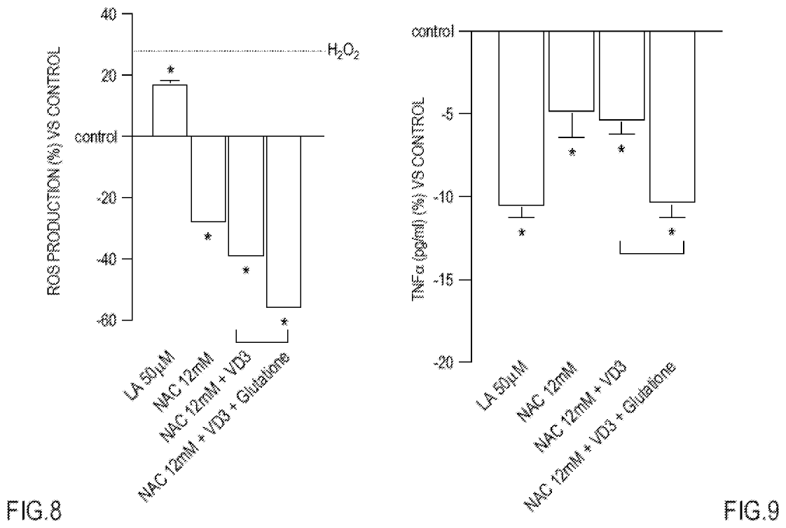

An absorption assay was used to evaluate the actual absorption at the encephalic level of N-acetylcysteine. The results, shown in FIG. 7 confirm improved efficacy of NAC when administered in combination with VitD. It was also observed that the presence of glutathione (NAC+VitD+G) further enhances this effect. Results designed to evaluate the antioxidant properties of LA, NAC, NAC+VitD and NAC+VitD+G are shown in FIG. 8.

Given the importance of oxygen radicals in neurodegenerative diseases and neuropathies, particularly peripheral neuropathies, tests were conducted in the presence of an oxidative state comparable to that due to aging/degeneration. The tested molecules confirmed their antioxidant activity. Combinations of NAC with VitD and with VitD+G show an amplified effect compared with NAC alone.

In addition, an evaluation of the anti-inflammatory activity of LA, NAC, NAC+VitD, and NAC+VitD+G was conducted. The evaluation was performed using a marker related to one of the main markers involved in the inflammatory process, namely TNFα. The marker results reduced, in particular, following treatment with NAC+VitD+G (FIG. 9).

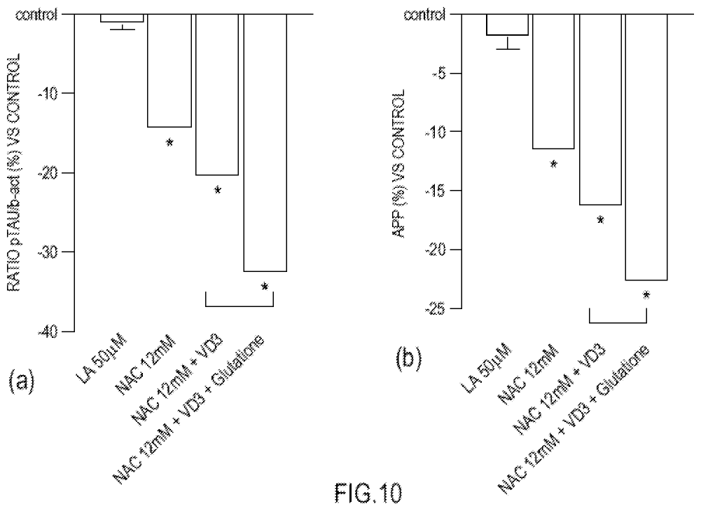

FIGS. 10(a) and 10(b) show the results obtained in a test evaluating the response of the intracellular markers TAU (FIG. 10(a)) and APP (FIG. 10(b)), following different stimulations. With reference to Tau_ (marker of cognitive decline), the data support the hypothesis of prevention of both physiological (aging) and potentially pathological (degeneration) cognitive degradation by the products tested. NAC in combination with VitD, and NAC in combination with VitD and G show the best biological profiles. Also in the case of the APP (beta-amyloid precursor) marker, NAC in combination with VitD, and NAC in combination with VitD and G show the best biological profiles. The data in FIG. 10(a) and FIG. 10(b) show that NAC in combination with VitD, and NAC in combination with VitD and G are effective in preventing both physiological (aging) and potentially pathological (degeneration) cognitive degradation and show a positive effect on brain cell health and growth.

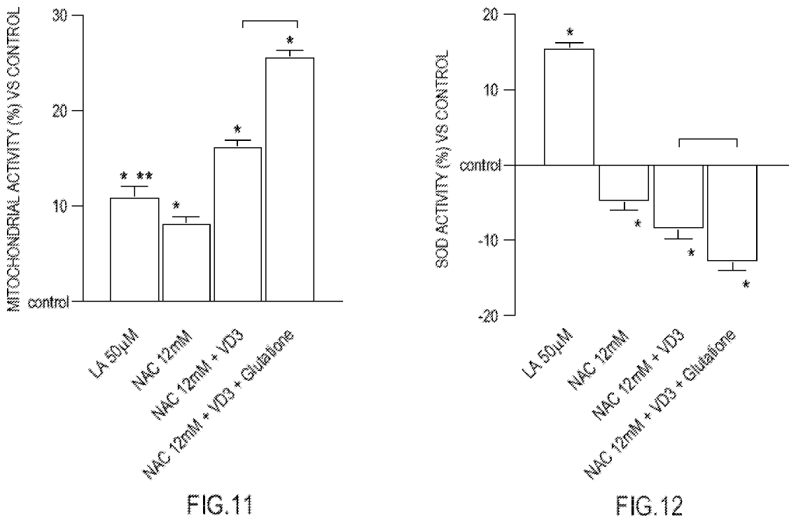

To investigate the effects of the composition of the invention at the peripheral nervous system level, mitochondrial viability and metabolism were initially assessed. The results of that test, shown in FIG. 11, show that the presence of VitD, or the VitD+G combination, contributes to increased mitochondrial activity.

The antioxidant effects of the composition of the invention, at the peripheral nervous system level, were investigated by an assay of SOD (superoxide dismutase) enzyme activity. The results (in FIG. 12) showed that the antioxidant activity of the molecules is maintained, suggesting a potential anti-inflammatory effect.

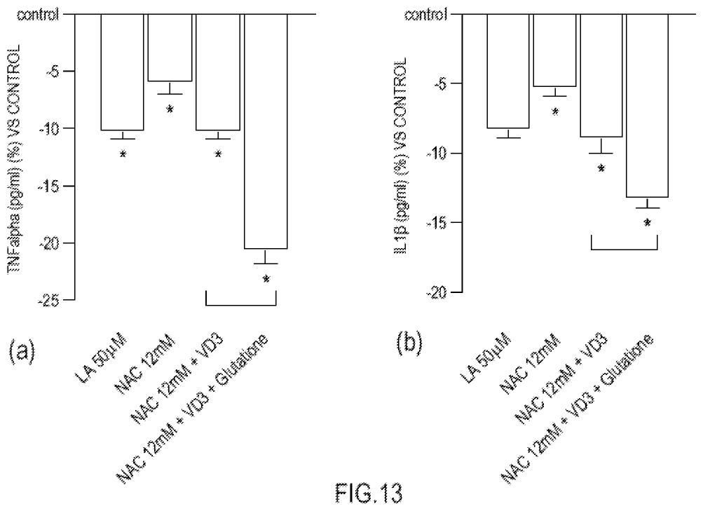

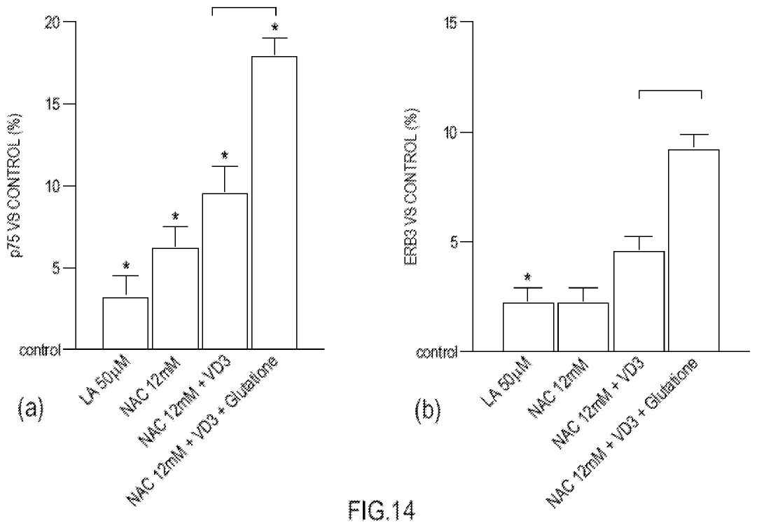

FIGS. 13(a) and 13(b) show the results obtained in a test to assess anti-inflammatory activity, at the level of the peripheral nervous system. The evaluation is performed on markers related to the two main mechanisms involved in the inflammatory response, namely TNFα (FIG. 13(a)) and IL1β (FIG. 13(b)). Inflammation constitutes a negative aspect of recovery after peripheral nerve injury. As can be seen from FIGS. 14(a) and 14(b), both inflammatory markers evaluated are reduced in the presence of the tested products. In particular, this reduction is particularly pronounced in the presence of NAC+VitD and NAC+VitD+G.

A test evaluating the proportion of lipoic acid or NAC absorbed (intra/extra ratio) at the level of the PNS showed that the ratio of the extra to the intra proportion confirms what has been observed, and that the combined (NAC+VitD and NAC+VitD+G) also reaches the peripheral nervous system after hepatic passage.

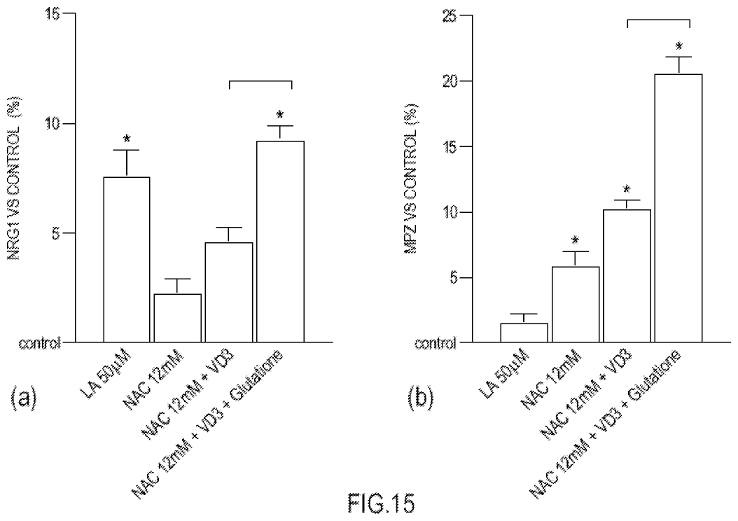

FIG. 15 shows the results of an analysis of intracellular markers related to the peripheral nervous system, specifically: p75, also known as p75NTR, (FIG. 15(a)) and ErbB3 (FIG. 15(b)). P75NTR has been identified as a positive modulator of Schwann cell myelination during development and is implicated in promoting nerve regeneration after injury. ErbB3 is a mediator of Schwann cell biological activity, and is implicated in the regulation of myelination, migration and axonal sorting. NRG1 is a regulator of Schwann cell myelination, while MPZ is an important molecule for the formation of compact myelin around neurites. The results obtained, with reference to all markers evaluated, show that the best effect is obtained by using NAC in combination with VitD and glutathione (G).

### Discussion of Results

The above experimental results demonstrate that the combination of N-acetylcysteine (NAC), vitamin D3 (vitD) and/or vitamin D2 and, optionally, glutathione, i.e., the composition of the invention, produces numerous beneficial effects. The combined effect of N-acetylcysteine with vitamin D and, preferably, glutathione, results in equal or greater benefits than those obtained by using lipoic acid, using N-acetylcysteine (in combination with vitamin D and, optionally glutathione) as a substitute for lipoic acid, thus avoiding the possible side effects of lipoic acid.

1. A composition comprising:
(i) a mixture that comprises or alternatively consists of:
(I) N-acetylcysteine or a pharmaceutically or food grade acceptable salt thereof or derivative thereof;
(II) a vitamin of the D group or a derivative thereof; and, preferably,
(III) glutathione or a derivative thereof; and optionally,
(ii) at least one food or pharmaceutical grade additive and/or excipient.

2. The composition according to claim 1, wherein said (II) vitamin of the D group is a vitamin D3 or cholecalciferol and/or vitamin D2 or a derivative thereof.

3. The composition according to claim 1, wherein said mixture (I) comprises N-acetylcysteine, or a salt or derivative thereof, and glutathione, or a salt or derivative thereof, in a weight ratio from 1:6 to 6:1, preferably from 1:5 to 5:1, more preferably from 1:4 to 4:1, even more preferably from 1:3 to 3:1, e.g. from 1:2 to 2:1, or 1:1.

4. The composition according to claim 1, wherein said mixture (i) comprises or alternatively consists of:
said (I) N-acetylcysteine in an amount by weight from 10% to 90%, preferably from 30% to 70%, with respect to the total weight of the mixture;
said (II) vitamin of the D group in an amount by weight from 0.0001% to 10%, preferably from 0.001% to 3%, with respect to the total weight of the mixture;
said (III) glutathione in an amount by weight from 15% to 85%, preferably from 20% to 75%, with respect to the total weight of the mixture.

5. A method of treatment comprising administering a composition of claim 1 to a subject that has a peripheral neuropathy a neurodegenerative disease or another disease.

6. The method of claim 5, wherein the subject has a neurodegenerative disease.

7. The method according to claim 6, wherein said neurodegenerative disease is selected from: Alzheimer's disease (AD), Parkinson's disease (PD), Huntington's disease, amyotrophic lateral sclerosis (ALS), frontotemporal dementia (FTD or presenile dementia), progressive supranuclear palsy (PSP or Steele-Richardson-Olszewski syndrome), Lewy body dementia (DLB), Creutzfeldt-Jakob disease (MCJ), and Gerstmann-Sträussler-Scheinker disease (GSS).

8. The method according to claim 5, wherein the subject has a neuropathy selected from: diabetic neuropathy, carpal, tarsal, ulnar, radial tunnel syndrome, cervicobrachialgia, herpes zoster, herniated intervertebral disc, sciatic neuralgia or lumbosciatica, Guillain-Barre syndrome, toxic and/or deficiency neuropathies, preferably drug neuropathy and alcoholic neuropathy, and infectious neuropathies, preferably HIV infection neuropathy and cytomegalovirus neuropathy.

9. The method of claim 5, wherein the subject has tumor, insulin resistance, metabolic disorders, obesity, thrombotic/thromboembolic diseases, ischemic episodes from surgery, transplantation, bypass, endometriosis, infertility, polycystic ovary syndrome (PCOS), polyabortion, inflammation, and oxidative stress.

10. The method of claim 5, wherein said composition comprising (I) and (II) and, preferably, (III) is administered in an effective amount, at least once or twice a day, so as to ensure administration to subjects in need thereof of daily dosages in the ranges:
from 50 mg to 2,000 mg, preferably from 100 mg to 2,000 mg, even preferably from 200 mg to 1,500 mg, more preferably from 300 mg to 1,000 mg, e.g. 150 mg, or 250 mg, or 400 mg, or 500 mg, or 600 mg, or 700 mg, or 800 mg, or 900 mg, of (I) N-acetylcysteine;
from 2 micrograms (mcg) to 4,000 mcg, preferably from 2 micrograms (mcg) to 2,500 mcg, even preferably from 2 micrograms (mcg) to 1,000 mcg, more preferably from 50 mcg to 800 mcg, even more preferably from 100 mcg to 600 mcg, e.g. 200 mcg, or 300 mcg, or 400 mcg, or 500 mcg, or 1,000 mcg of vitamin D, preferably vitamin D3;
from 10 mg to 2,000 mg, preferably from 50 mg to 1,500 mg, more preferably from 100 mg to 1,200 mg, e.g., 200 mg, or 300 mg, or 400 mg, or 500 mg, or 600 mg, or 700 mg, or 800 mg, or 900 mg, or 1,000 mg, of (III) glutathione.

11. The method of claim 5, wherein said composition is for oral use in said subject in need thereof.

12. Non-therapeutic use of the composition according to claim 1, wherein said composition is for use in a, preventive and/or curative, method of non-therapeutic treatment of a peripheral neuropathy and/or symptoms or disorders associated with said peripheral neuropathy.

13. Non-therapeutic use of the composition according to claim 1, wherein said composition is for use in a method of non-therapeutic treatment of a symptom or disorder, preferably associated with brain aging in a subject in need thereof.

14. Non-therapeutic use of the composition according to claim 1, wherein said composition is for use in a method of non-therapeutic treatment of slowing down brain aging in a subject in need thereof.
